(19) United States
(12) Patent Application Publication
(10) Pub. No.: US 2020/0030278 A1
(43) Pub. Date: Jan. 30, 2020

(54) PHARMACEUTICAL COMPOSITIONS OF METAP-2 INHIBITORS

(71) Applicant: Zafgen, Inc., Boston, MA (US)

(72) Inventors: James E. Vath, Lynnfield, MA (US); Bryan Burkey, Ipswich, MA (US)

(21) Appl. No.: 16/484,600

(22) PCT Filed: Feb. 12, 2018
(86) PCT No.: PCT/US2018/017778

§ 371 Date: Aug. 8, 2019

Related U.S. Application Data
(60) Provisional application No. 62/457,355, filed on Feb. 10, 2017.
(60) Provisional application No. 62/457,347, filed on Feb. 10, 2017.

(51) Int. Cl. A61K 31/336; A61K 31/5377; A61K 9/00

(52) U.S. Cl. CPC A61K 31/336; A61K 9/0019; A61K 31/5377

(57) ABSTRACT

Disclosed herein, in part, are pharmaceutical compositions comprising a MetAp-2 inhibitor and a pharmaceutically acceptable excipient. The pharmaceutical compositions are contemplated to be useful, for example in the treatment of obesity.

### CROSS REFERENCE TO RELATED APPLICATIONS

This application is a national stage filing under 35 U.S.C. § 371 of PCT/US2018/017778, filed Feb. 12, 2018, which claims the benefit of, and priority to, U.S. Provisional Patent Application No. 62/457,347, filed Feb. 10, 2017, and U.S. Provisional Patent Application No. 62/457,355, filed Feb. 10, 2017, the contents of each of which are hereby incorporated by reference in their entirety.

### BACKGROUND

MetAP-2 encodes a protein that functions at least in part by enzymatically removing the amino terminal methionine residue from certain newly translated proteins such as glyceraldehyde-3-phosphate dehydrogenase (Warder et al. (2008) J. Proteome Res. 7:4807). Increased expression of the MetAP-2 gene has been historically associated with various forms of cancer. Molecules inhibiting the enzymatic activity of MetAP-2 have been identified and have been explored for their utility in the treatment of various tumor types (Wang et al. (2003) Cancer Res. 63:7861) and infectious diseases such as microsporidiosis, leishmaniasis, and malaria (Zhang et al. (2002) J. Biomed. Sci. 9:34). Notably, inhibition of MetAP-2 activity in obese and obese-diabetic animals leads to a reduction in body weight in part by increasing the oxidation of fat and in part by reducing the consumption of food (Rupnick et al. (2002) Proc. Natl. Acad. Sci. USA 99:10730).

Such MetAP-2 inhibitors may be useful as well for patients with excess adiposity and conditions related to adiposity including type 2 diabetes, hepatic steatosis, and cardiovascular disease (via e.g. ameliorating insulin resistance, reducing hepatic lipid content, and reducing cardiac workload). For example, over 1.1 billion people worldwide are reported to be overweight.

The MetAP2 inhibitor beloranib has produced consistent and clinically meaningful weight loss in clinical trials of patients with obesity, type 2 diabetes, Prader-Willi syndrome (PWS), and hypothalamic injury-associated obesity. In patients with type 2 diabetes, beloranib produced 13% weight loss and a 2.0% reduction in HbA1c over 26 weeks of treatment. Beloranib was generally well tolerated in preclinical testing. However, in clinical trials of beloranib in patients with obesity, PWS, or type 2 diabetes, adverse events (AEs) of venous thromboembolism occurred in beloranib-treated patients despite being otherwise generally well-tolerated. These AEs included superficial thrombophlebitis, deep vein thrombosis, and pulmonary embolism (PE), including two fatal PEs in patients with PWS that resulted in cessation of beloranib development.

There is a need for METAP-2 formulations that have an improved safety profile compared to beloranib while retaining similar metabolic efficacy in models of obesity and cardiometabolic disease.

### SUMMARY

The present disclosure provides, for example, pharmaceutical compositions comprising a MetAP-2 inhibitor as an active ingredient as well as provides for their use as medicaments and/or in the manufacture of medicaments for the inhibition of MetAP-2 activity in warm-blooded animals such as humans. In particular this disclosure relates to pharmaceutical compositions useful for the treatment of obesity, type 2 diabetes, and other obesity-associated conditions. A disclosed pharmaceutical composition may comprise, for example, at least one MetAP-2 inhibitor and a pharmaceutically acceptable carrier.

In an embodiment, a pharmaceutically acceptable composition comprising a MetAP-2 inhibitor and a pharmaceutically acceptable excipient is provided, said composition providing at least one of, for example: a volume of distribution at steady state (VDₛₛ) of less than about 5 L/kg, a Tₘₐₓ of less than about 0.5 hr and/or Cₘₐₓ of more than about 10 ng/mL upon subcutaneous administration to a human patient.

For example, provided herein is a pharmaceutically acceptable composition, comprising a MetAP-2 inhibitor and a pharmaceutically acceptable excipient, that has a very short half-life upon administration, which may avoid significant side effects while still being efficacious.

In another embodiment, a pharmaceutically acceptable composition comprising a MetAP-2 inhibitor and a pharmaceutically acceptable excipient is provided, wherein the half-life of the MetAP-2 inhibitor upon subcutaneous administration to a human patient is about, for example, 10 times less than the half-life of beloranib when administered, e.g. intravenously, to a human patient.

Methods of treating and/or controlling obesity are contemplated herein, comprising administering to a patient in need thereof an effective amount of a disclosed pharmaceutical composition. In an embodiment, a method of inducing weight loss in a patient in need thereof is provided, comprising administering to said patient an effective amount of a disclosed pharmaceutical composition. In another embodiment, a method of substantially preventing weight gain in a patient in need thereof is provided, comprising administering to said patient an effective amount of a disclosed pharmaceutical composition.

Methods of treating obesity in a patient in need thereof are also contemplated herein, comprising, for example: administering a MetAP-2 inhibitor; monitoring the patient at discrete intervals for risk of a thromboembolic event; and determining at the discrete intervals that the patient has an indication identifying the patient is at particular risk of a thromboembolic event upon treatment with the MetAP-2 inhibitor.

### BRIEF DESCRIPTION OF THE DRAWINGS

FIG. 1 depicts plasma concentration profiles of beloranib and Compound A in rats, dogs, rabbits and humans after a single subcutaneous dose (linear scale).

FIG. 2 depicts plasma concentration profiles of beloranib and Compound A in rats, dogs, rabbits and humans after a single subcutaneous dose (log-linear scale).

FIG. 3 depicts relative concentrations of radioactivity derived from [¹⁴C] beloranib (6 mg/kg) or [¹⁴C]Compound A (1 mg/kg) in testes after subcutaneous administration in rats. Concentrations of Compound A have been normalized to a 1 mg/kg dose.

FIG. 4 depicts relative concentrations of radioactivity derived from [¹⁴C] beloranib (6 mg/kg) or [¹⁴C]Compound A (1 mg/kg) in bone and white fat after subcutaneous administration in rats. Concentrations of Compound A have been normalized to a 1 mg/kg dose.

FIG. 5 depicts relative concentrations of radioactivity derived from [¹⁴C] beloranib (6 mg/kg) or [¹⁴C]Compound A (1 mg/kg) in liver and kidney after subcutaneous administration in rats.

FIG. 6 depicts mean and SD plasma Compound A and beloranib concentrations following acute s.c. administration in dogs (n=8-10/group). Compound A was undetectable in plasma after the 4-hour timepoint in dogs.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

The term “alkoxyalkyl” as used herein refers to a straight or branched alkyl group attached to oxygen, attached to a second straight or branched alkyl group (alkyl-O-alkyl-). Exemplary alkoxyalkyl groups include, but are not limited to, alkoxyalkyl groups in which each of the alkyl groups independently contains 1-6 carbon atoms, referred to herein as C₁₋₆alkoxy-C₁₋₆alkyl. Exemplary alkoxyalkyl groups include, but are not limited to methoxymethyl, 2-methoxyethyl, 1-methoxyethyl, 2-methoxypropyl, ethoxymethyl, 2-isopropoxyethyl etc.

The term “alkyoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkenyloxy” used herein refers to a straight or branched alkenyl group attached to oxygen (alkenyl-O—). Exemplary alkenyloxy groups include, but are not limited to, groups with an alkenyl group of 3-6 carbon atoms, referred to herein as C₃₋₆alkenyloxy. Exemplary “alkenyloxy” groups include, but are not limited to allyloxy, butenyloxy, etc.

The term “alkynyloxy” used herein refers to a straight or branched alkynyl group attached to oxygen (alkynyl-O). Exemplary alkynyloxy groups include, but are not limited to, groups with an alkynyl group of 3-6 carbon atoms, referred to herein as C₃₋₆alkynyloxy. Exemplary alkynyloxy groups include, but are not limited to, propynyloxy, butynyloxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₁₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “cyano” as used herein refers to the radical —CN.

The term “cycloalkoxy” as used herein refers to a cycloalkyl group attached to oxygen (cycloalkyl-O—). Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc

The terms “cycloalkyl” or a “carbocyclic group” as used herein refers to a saturated or partially unsaturated hydrocarbon group of, for example, 3-6, or 4-6 carbons, referred to herein as C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

The term “heterocyclyloxy” as used herein refers to a heterocyclyl group attached to oxygen (heterocyclyl-O—).

The term “heteroaryloxy” as used herein refers to a heteroaryl group attached to oxygen (heteroaryl-O—).

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound (e.g., a MetAP-2 inhibitor) as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The pharmaceutical compositions of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment of obesity or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The pharmaceutical compositions of the present disclosure are administered in therapeutically effective amounts to treat a disease. Alternatively, a therapeutically effective amount of a pharmaceutical composition is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds included in the compositions of the present disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds included in the compositions of the present disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds included in the compositions of the present disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds included in the compositions of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds included in the compositions of the present disclosure can exist in solvated as well as unsolvated forms with pharmaceutically acceptable solvents such as water, ethanol, and the like, and it is intended that the present disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The present disclosure also embraces isotopically labeled compounds included in the compositions of the present disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, 35S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the present disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as β-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound included in the compositions of the present disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound included in the compositions of the present disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

### I. Pharmaceutical Compositions

In an embodiment, the present disclosure provides for pharmaceutically acceptable compositions comprising a MetAP-2 inhibitor, and a pharmaceutically acceptable excipient, where the composition provides at least one of: a volume of distribution at steady state (VDₛₛ) of less than about 5 L/kg, a Tₘₐₓ of less than about 0.5 hr and/or Cₘₐₓ of more than about 10 ng/mL upon subcutaneous administration to a human patient.

In certain embodiments, a contemplated pharmaceutically acceptable composition may provide, for example, a half life (t_{1/2}) of less than 1 hour upon subcutaneous administration to the patient.

In certain other embodiments, a contemplated composition pharmaceutically acceptable may provide, for example, less than a plasma concentration of 1 ng/mL about 8 hours after administration to a patient, e.g., a human patient, or less than a plasma concentration of 0.15 ng/mL, or e.g. about 0.0 (e.g., undectatable) to about 0.2 ng/mL or less than a plasma concentration of about 0.15 ng/mL, or e.g., about 0.12 ng/mL at 8 hours (or e.g. at 4, 6, 12 hours) after administration to a human patient.

For example, provided herein are pharmaceutically acceptable compositions comprising a MetAP-2 inhibitor, such as those disclosed herein, and a pharmaceutically acceptable excipient, wherein the composition upon administration to patient (e.g., a human patient) provides a plasma concentration at about 8 hours after administration that is less than than EC₅₀ value of the compound in the composition as tested against human venous endothelial cell (HUVEC) proliferation (as tested in HUVEC cells for example at 72 hours).

Another aspect of the present disclosure provides pharmaceutically acceptable compositions comprising a MetAP-2 inhibitor and a pharmaceutically acceptable excipient, wherein the half-life of the MetAP-2 inhibitor upon subcutaneous administration to a human patient may be, for example, about 10 times less than the half-life of, e.g., beloranib when intravenously administered to a human patient.

In certain embodiments, a contemplated MetAP-2 inhibitor included in the compositions of the present disclosure may be an irreversible inhibitor. In certain embodiments, the irreversible inhibitor may covalently bind, for example, to His231 of MetAP-2 via, e.g., a spiro epoxide moiety present on the irreversible inhibitor.

For example, a contemplated MetAP-2 inhibitor included in the compositions of the present disclosure may be an analog of, e.g., fumagillin.

In certain embodiments, a contemplated MetAP-2 inhibitor included in the compositions of the present disclosure may be represented by:

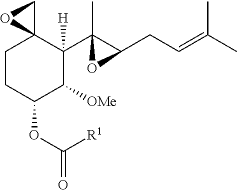

wherein R¹ may selected from C₁₋₈alkylene, C₂₋₈alkenylene, heterocyclyl, C₃₋₆cycloalkyl, —NR^{a}—C₁₋₈alkylene, —NR^{a}—C₂₋₈alkenylene, and —NR^{a}—C₃₋₆cycloalkyl; wherein R¹ may be substituted by a substituent selected from the group consisting of: carboxy, —O—C(O)—NR^{a}R^{b}, —C(O)—O—C₁₋₆alkyl, phenyl (optionally substituted by substituent selected from NR^{a}R^{b}, C₁₋₆alkoxy (optionally substituted by a substituent selected from the group consisting of NR^{a}R^{b}, C₁₋₆alkyl, and heterocyclic)), C₁₋₆alkylene (optionally substituted by hydroxyl, heterocycyl, NR^{a}R^{b}, carboxy, and —C(O)—O—C₁₋₆alkyl); wherein R^{a} and R^{b} are each independently selected from hydrogen and C₁₋₆alkyl, or R^{a} and R^{b} together with the nitrogen to which they are attached may form a 4-7 membered heterocyclic ring;

and pharmaceutically acceptable salts, stereoisomers, esters and prodrugs thereof.

In certain other embodiments, a contemplated MetAP-2 inhibitor included in the compositions of the present disclosure may be represented by Formula II:

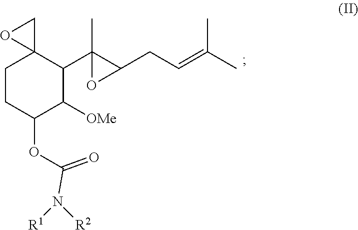

wherein:

R¹ and R², together with the nitrogen to which they are attached, form a 4-6 membered saturated heterocyclic ring A, or a 6-8 membered bicyclic, fused, bridged or spirocyclic hetercyclic ring A, where ring A which may have an additional heteroatom selected from the group consisting of O, S(O)_{w} (wherein w is 0, 1, or 2), and NR^{a};

heterocyclic ring A is substituted on an available carbon by a substituent represented by L-B; and wherein heterocyclic ring A is additionally and optionally substituted by one or two substituents each independently selected from the group consisting of halogen, hydroxyl, C₁₋₃alkyl and C₁₋₃alkoxy; wherein C₁₋₃alkyl and C₁₋₃alkoxy may optionally be substituted by one or more fluorine atoms or a substituent selected from the group consisting of cyano, hydroxyl, and N(R^{a}R^{b});

L is selected from the group consisting of C₁₋₆alkylene and C₁₋₆alkenylene; wherein C₁₋₆alkylene and C₁₋₆alkenylene may optionally be substituted by one or two substituents each independently selected from the group consisting of halogen and hydroxyl; and wherein one or two methylene units of L may optionally and independently be replaced by a moiety selected from the group consisting of a bond, —O—, —C(O)—, —O—C(O)—, —C(O)—O—, —NR^{a}—, —C(O)—NR^{a}—, —NR^{a}—C(O)—, —O—C(O)—NR^{a}—, —NR^{a}—C(O)—O—, —S(O)_{w}— (wherein w is 0, 1, or 2), —S(O)_{w}—NR^{a}—, and —NR^{a}—S(O)_{w}—;

B is selected from the group consisting of RⁱR^{j}N—, heterocyclyl, heterocyclyloxy, heteroaryl, heterocyclyl-(NR^{a})—, and hydrogen; wherein said heteroaryl may optionally be substituted with one or more substituents selected from R^{f}; and wherein said heterocyclyl is bound to L through a ring carbon and may optionally be substituted by one or more substituents selected from R^{g}; and wherein if said heterocyclyl contains a —NH moiety that nitrogen may optionally be substituted by R^{h};

Rⁱ and R^{j} are selected independently for each occurrence from the group consisting of hydrogen, C₁₋₆alkyl, C₂₋₆alkenyl, C₃₋₆cycloalkyl, heterocyclyl and heterocyclylcarbonyl; wherein C₁₋₆alkyl, C₂₋₆alkenyl and C₃₋₆cycloalkyl may be optionally substituted by one or more substituents independently selected from the group consisting of fluorine, hydroxyl, cyano, R^{a}R^{b}N—, R^{a}R^{b}N-carbonyl- and C₁₋₃alkoxy; and wherein heterocyclyl and heterocyclylcarbonyl may be optionally substituted by one or more substituents independently selected from the group consisting of C₁₋₆alkyl, C₂₋₆alkenyl, C₂₋₆alkynyl, C₃₋₆cycloalkyl, C₁₋₆alkoxy, halo-C₁₋₆-alkyl, hydroxyl-C₁₋₆-alkyl, R^{a}R^{b}N—C₁₋₆alkyl- and C₁₋₆-alkoxy-C₁₋₆-alkyl group; and wherein if said heterocyclyl or heterocyclylcarbonyl contains a —NH moiety that nitrogen may optionally be substituted by one or more groups independently selected from the group consisting of C₁₋₆alkyl, C₃₋₆alkenyl, C₃₋₆alkynyl, C₃₋₆cycloalkyl, C₁₋₆alkyl-S(O)₂— and C₁₋₆-alkylcarbonyl;

or Rⁱ and R^{j} taken together with the nitrogen to which they are attached form a 4-9 membered monocyclic, bridged bicyclic, fused bicyclic or spirocyclic heterocyclic ring, which may have an additional heteroatom selected from the group consisting of O, N, and S(O)_{w} (wherein w is 0, 1 or 2); wherein the 4-9 membered monocyclic, bridged bicyclic, fused bicyclic or spirocyclic heterocyclic ring may be optionally substituted on carbon by one, two, or more substituents selected from the group consisting of halogen, hydroxyl, oxo, cyano, C₁₋₆alkyl, C₁₋₆alkoxy, R^{a}R^{b}N—, R^{a}R^{b}N—SO₂— and R^{a}R^{b}N-carbonyl-; wherein said C₁ 6alkyl or C₁₋₆alkoxy may optionally be substituted the group consisting of fluorine, hydroxyl, and cyano; and wherein if said 4-9 membered monocyclic, bridged bicyclic, fused bicyclic or spirocyclic heterocyclic ring contains a —NH moiety that nitrogen may be optionally substituted by a substituent selected from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, and C₁₋₆alkoxycarbonyl- may optionally be substituted by one or more substituents selected from the group consisting of fluorine, hydroxyl, and cyano;

R^{a} and R^{b} are independently selected, for each occurrence, from the group consisting of hydrogen and C₁₋₃alkyl; wherein C₁₋₃alkyl may optionally be substituted by one or more substituents selected from halogen, cyano, oxo and hydroxyl;

R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, C₁₋₆alkoxycarbonyl-N(R^{a})— may be optionally substituted by one or more substituents selected from R^{P};

R^{g} is independently selected for each occurrence from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}— (wherein w is 0, 1 or 2), C₁₋₆alkylcarbonyl-N(R^{a})— and C₁₋₆alkoxycarbonyl-N(R^{a})—; wherein C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};

R^{h} is independently selected for each occurrence from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, and C₁₋₆alkoxycarbonyl- may optionally be substituted by one or more substituents selected from R^{P}; and

R^{P} is independently selected, for each occurrence, from the group consisting of halogen, hydroxyl, cyano, C₁₋₆alkoxy, RⁱR^{j}N—, RⁱR^{j}N-carbonyl-, RⁱR^{j}N—SO₂—, and RⁱR^{j}N-carbonyl-N(R^{a});

and pharmaceutically acceptable salts, stereoisomers, esters and prodrugs thereof.

Other contemplated MetAP-2 inhibitors include those represented by Formula III or IIIa:

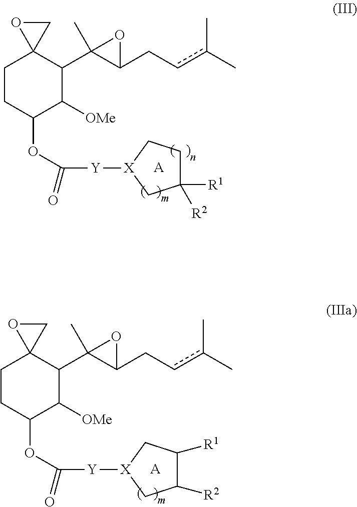

wherein:

[symbol] is a single or double bond;

Y is a bond or NR^{a};

X is N or CR^{N}; wherein X is N when Y is a bond and X is CR^{N} when Y is NR^{a};

n is 0 or 1;

m is 1 or 2;

Ring A may be optionally substituted by one or two substituents each independently selected from the group consisting of halogen, hydroxyl, C₁₋₃alkyl and C₁₋₃alkoxy, wherein C₁₋₃alkyl and C₁₋₃alkoxy may optionally be substituted by one or more fluorine atoms or a substituent selected from the group consisting of cyano, hydroxyl, and N(R^{a}R^{b});

R¹ and R², together with the carbon or carbons to which they are attached, form a 4-6 membered saturated heterocyclic ring B having one or two heteroatoms selected from the group consisting of O, S(O)_{w} (wherein w is 0, 1 or 2) and NR^{h} or form a 3-6 membered saturated carbocyclic ring B; wherein the heterocyclic or carbocyclic ring B may optionally be substituted on a free carbon by one or two substituents each independently selected from the group consisting of halogen, hydroxyl, oxo, C₁₋₃alkyl, C₁₋₃alkoxy, —C(O)—NRⁱR^{j}, —C(O)—N(R^{a})—C₁₋₆alkylene-NRⁱR^{j}, —C₁₋₆alkylene-NRⁱR^{j}, —C₁₋₆alkylene-O—C(O)—NRⁱR^{j}, and —O—C(O)—NRⁱR^{j}; wherein C₁₋₃alkyl, C₁₋₃alkoxy, —C(O)—NRⁱR^{j}, —C(O)—N(R^{a})—C₁₋₆alkylene-NRⁱR^{j}, —C₁₋₆alkylene-NRⁱR^{j}, —C₁₋₆alkylene-O—C(O)—NRⁱR^{j}, and —O—C(O)—NRⁱR^{j} may optionally be substituted by one or more fluorine atoms or a group selected from cyano, hydroxyl, or N(R^{a}R^{b});

Rⁱ and R^{j} are selected independently for each occurrence from the group consisting of hydrogen, C₁₋₆alkyl, C₂₋₆alkenyl, C₃₋₆cycloalkyl, heterocyclyl and heterocyclylcarbonyl; wherein C₁₋₆alkyl, C₂₋₆alkenyl and C₃₋₆cycloalkyl may be optionally substituted by one or more substituents independently selected from the group consisting of fluorine, hydroxyl, cyano, R^{a}R^{b}N—, R^{a}R^{b}N-carbonyl- and C₁₋₃alkoxy; and wherein heterocyclyl and heterocyclylcarbonyl may be optionally substituted by one or more substituents independently selected from the group consisting of C₁₋₆alkyl, C₂₋₆alkenyl, C₂₋₆alkynyl, C₃₋₆cycloalkyl, C₁₋₆alkoxy, halo-C₁₋₆-alkyl, hydroxyl-C₁₋₆-alkyl, R^{a}R^{b}N—C₁₋₆alkyl- and C₁₋₆-alkoxy-C₁₋₆-alkyl; and wherein if said heterocyclyl or heterocyclylcarbonyl contains a —NH moiety that nitrogen may optionally be substituted by one or more groups independently selected from the group consisting of C₁₋₆alkyl, C₃₋₆alkenyl, C₃₋₆alkynyl, C₃₋₆cycloalkyl, C₁₋₆alkyl-S(O)₂— and C₁₋₆-alkylcarbonyl;

or Rⁱ and R^{j} taken together with the nitrogen to which they are attached form a 4-9 membered heterocyclic ring, which may have an additional heteroatom selected from the group consisting of N, O, and S(O)_{w} (wherein w is 0, 1 or 2); wherein the heterocyclic ring may be optionally substituted on carbon by one, two, or more substituents selected from the group consisting of halogen, hydroxyl, oxo, cyano, C₁₋₆alkyl, C₁₋₆alkoxy, R^{a}R^{b}N—, R^{a}R^{b}N—SO₂— and R^{a}R^{b}N-carbonyl-; wherein said C₁₋₆alkyl and C₁₋₆alkoxy may optionally be substituted the group consisting of fluorine, hydroxyl, and cyano; and wherein if said heterocyclic ring contains a —NH moiety that nitrogen may be optionally substituted by a substituent selected from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁ 6alkyl-S(O)₂—, C₁₋₆alkylcarbonyl-, and C₁₋₆alkoxycarbonyl- may optionally be substituted by one or more substituents selected from the group consisting of fluorine, hydroxyl, and cyano;

R^{h} is independently selected for each occurrence from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, and C₁₋₆alkoxycarbonyl- may optionally be substituted by one or more substituents selected from R^{P};

R^{P} is independently selected, for each occurrence, from the group consisting of halogen, hydroxyl, cyano, C₁₋₆alkoxy, RⁱR^{j}N—, RⁱR^{j}N-carbonyl-, RⁱR^{j}N—SO₂—, and RⁱR^{j}N-carbonyl-N(R^{a})—;

R^{N} is selected from the group consisting of hydrogen, halogen, hydroxyl, and C₁₋₆alkyl; and

R^{a} and R^{b} are independently selected, for each occurrence, from the group consisting of hydrogen and C₁₋₁₄alkyl; wherein C₁₋₁₄alkyl may optionally be substituted by one or more substituents selected from the group consisting of halogen, cyano, oxo, and hydroxyl; and pharmaceutically acceptable salts, stereoisomers, esters, and prodrugs thereof.

For example, a contemplated MetAP-2 inhibitor included in the compositions of the present disclosure may be selected from the group consisting of, e.g., (3R,4S,5S,6R)-5-methoxy-4-((2R,3R)-2-methyl-3-(3-methylbut-2-en-1-yl)oxiran-2-yl)-1-oxaspiro[2.5]octan-6-yl (E)-3-(4-(2-(dimethylamino)ethoxy)phenyl)acrylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-morpholinoethyl)azetidine-1-carboxylate (Compound A), (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-(3,3-difluoroazetidin-1-yl)ethyl)azetidine-1-carboxylate, (3R,4S,5S,6R)-4-((2R,3R)-3-isopentyl-2-methyloxiran-2-yl)-5-methoxy-1-oxaspiro[2.5]octan-6-yl 3-hydroxy-3-methylazetidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-hydroxy-3-methylazetidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl ((S)-2-methyl-1-(1-(((R)-1-methylpyrrolidin-3-yl)methyl)-1H-imidazol-4-yl)propyl)carbamate, (3R,4S,5S,6R)-5-methoxy-4-((2R,3R)-2-methyl-3-(3-methylbut-2-en-1-yl)oxiran-2-yl)-1-oxaspiro[2.5]octan-6-yl (S)-3-(2-morpholinoethyl)pyrrolidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 7-oxa-2-azaspiro[3.5]nonane-2-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl (3 aR,6aS)-tetrahydro-1H-furo[3,4-c]pyrrole-5(3H)-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl (3aR,6aS)-5-(2,2-difluoroethyl)hexahydropyrrolo[3,4-c]pyrrole-2(1H)-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-(1H-pyrazol-1-yl)ethyl)azetidine-1-carboxylate, (4S,5S,6R)-5-methoxy-4-((2R,3R)-2-methyl-3-(3-methylbut-2-en-1-yl)oxiran-2-yl)-1-oxaspiro[2.5]octan-6-yl 3-(2-(8-oxa-3-azabicyclo[3.2.1]octan-3-yl)ethyl)azetidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-azabicyclo[3.1.0]hexane-3-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 6-(morpholinomethyl)-2-azaspiro[3.3]heptane-2-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 6-(2,2-difluoroethyl)-2,6-diazaspiro[3.3]heptane-2-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-((dimethylcarbamoyl)oxy)azetidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-((ethylcarbamoyl)oxy)azetidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 6-morpholino-2-azaspiro[3.3]heptane-2-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl (R)-3-((dimethylcarbamoyl)oxy)pyrrolidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl (S)-3-((dimethylcarbamoyl)oxy)pyrrolidine-1-carboxylate, (3R,4S,5S,6R)-5-methoxy-4-((2R,3R)-2-methyl-3-(3-methylbut-2-en-1-yl)oxiran-2-yl)-1-oxaspiro[2.5]octan-6-yl 4-((dimethylcarbamoyl)oxy)piperidine-1-carboxylate, and pharmaceutically acceptable salts or stereoisomers thereof.

In an embodiment, a contemplated MetAP-2 inhibitor included in the compositions of the present disclosure, upon administration, may degrade to metabolites resulting from one or more metabolic pathways selected from the group consisting of CYP-mediated oxidation, GSH conjugation, and epoxide hydrolase-mediated hydrolysis.

Also provided herein is a compound represented by:

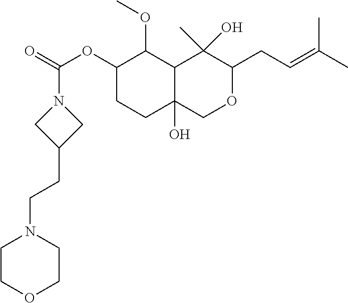 [SMILES: COC1C(OC(=O)N2CC(CCN3CCOCC3)C2)CCC2(O)COC(CC=C(C)C)C(C)(O)C12]

or a pharmaceutically acceptable salt or stereoisomer thereof.

Another aspect of the present disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more disclosed compounds, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the present disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present invention.

### II. Methods

Another aspect of the present disclosure provides methods of modulating the activity of MetAP-2. Such methods comprise administering to a patient in need thereof a pharmaceutically acceptable composition described herein. In some embodiments the compound(s) included in the compositions of the present disclosure may be one of the generic, subgeneric, or specific compounds described herein, such as a compound of Formula I. Another aspect of the present disclosure provides methods of treating a disease associated with expression or activity of MetAP2 in a patient.

In certain embodiments, the present disclosure provides a method of treating and/or controlling obesity in a patient in need thereof, comprising administering to the patient a disclosed pharmaceutically acceptable composition.

In certain embodiments, the present disclosure provides a method of inducing weight loss in a patient in need thereof, comprising administering to the patient a disclosed pharmaceutically acceptable composition.

In certain embodiments, the present disclosure provides a method of substantially preventing weight gain in a patient in need thereof, comprising administering to the patient a disclosed pharmaceutically acceptable composition.

In certain embodiments, the pharmaceutically acceptable composition may be administered to the patient daily, every other day, or two or three times a week.

In certain embodiments, the patient is a human.

In certain embodiments, the patient is a cat or dog.

In certain embodiments, the patient has a body mass index greater than or equal to about 30 kg/m² before the administration.

In certain embodiments, administering a disclosed pharmaceutically acceptable composition may comprise subcutaneous administration. In certain embodiments, administering a disclosed pharmaceutically acceptable composition may comprise intravenous administration.

Provided methods of treatment may include administering a disclosed pharmaceutically acceptable composition once, twice, or three times daily; about every other day (e.g. every 2 days); twice weekly (e.g. every 3 days, every 4 days, every 5 days, every 6 days, or e.g. administered with an interval of about 2 to about 3 days between doses); once weekly; three times weekly; every other week; twice monthly; once a month; every other month; or even less often.

Other contemplated methods of treatment include method of treating or ameliorating an obesity-related condition or co-morbidity, by administering a pharmaceutically acceptable composition disclosed herein to a subject. For example, contemplated herein are methods for treating type 2 diabetes in a patient in need thereof.

Exemplary co-morbidities include cardiac disorders, endocrine disorders, respiratory disorders, hepatic disorders, skeletal disorders, psychiatric disorders, metabolic disorders, and reproductive disorders.

Exemplary cardiac disorders include hypertension, dyslipidemia, ischemic heart disease, cardiomyopathy, cardiac infarction, stroke, venous thromboembolic disease and pulmonary hypertension. Exemplary endocrine disorders include type 2 diabetes and latent autoimmune diabetes in adults. Exemplary respiratory disorders include obesity-hypoventilation syndrome, asthma, and obstructive sleep apnea. An exemplary hepatic disorder is nonalcoholic fatty liver disease. Exemplary skeletal disorders include back pain and osteoarthritis of weight-bearing joints. Exemplary metabolic disorders include Prader-Willi Syndrome and polycystic ovary syndrome. Exemplary reproductive disorders include sexual dysfunction, erectile dysfunction, infertility, obstetric complications, and fetal abnormalities. Exemplary psychiatric disorders include weight-associated depression and anxiety.

In particular, in certain embodiments, the present disclosure provides a method of treating one or more of the above medical indications comprising administering to a subject in need thereof a therapeutically effective amount of a pharmaceutically acceptable composition described herein.

Obesity or reference to “overweight” refers to an excess of fat in proportion to lean body mass. Excess fat accumulation is associated with increase in size (hypertrophy) as well as number (hyperplasia) of adipose tissue cells. Obesity is variously measured in terms of absolute weight, weight:height ratio, distribution of subcutaneous fat, and societal and esthetic norms. A common measure of body fat is Body Mass Index (BMI). The BMI refers to the ratio of body weight (expressed in kilograms) to the square of height (expressed in meters). Body mass index may be accurately calculated using either of the formulas: weight(kg)/height²(m²) (SI) or 703×weight(lb)/height²(in²) (US).

In accordance with the U.S. Centers for Disease Control and Prevention (CDC), an overweight adult has a BMI of 25 kg/m² to 29.9 kg/m², and an obese adult has a BMI of 30 kg/m² or greater. A BMI of 40 kg/m² or greater is indicative of morbid obesity or extreme obesity. Obesity can also refer to patients with a waist circumference of about 102 cm for males and about 88 cm for females. For children, the definitions of overweight and obese take into account age and gender effects on body fat. Patients with differing genetic background may be considered “obese” at a level differing from the general guidelines, above.

The pharmaceutically acceptable compositions of the present disclosure also are useful for reducing the risk of secondary outcomes of obesity, such as reducing the risk of left ventricular hypertrophy. Methods for treating patients at risk of obesity, such as those patients who are overweight, but not obese, e.g. with a BMI of between about 25 and 30 kg/m², are also contemplated. In certain embodiments, a patient is a human.

BMI does not account for the fact that excess adipose can occur selectively in different parts of the body, and development of adipose tissue can be more dangerous to health in some parts of the body rather than in other parts of the body. For example, “central obesity”, typically associated with an “apple-shaped” body, results from excess adiposity especially in the abdominal region, including belly fat and visceral fat, and carries higher risk of co-morbidity than “peripheral obesity”, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, and a centrally obese adult typically has a WHR of about 0.85 or greater if female and about 0.9 or greater if male.

Methods of determining whether a subject is overweight or obese that account for the ratio of excess adipose tissue to lean body mass involve obtaining a body composition of the subject. Body composition can be obtained by measuring the thickness of subcutaneous fat in multiple places on the body, such as the abdominal area, the subscapular region, arms, buttocks and thighs. These measurements are then used to estimate total body fat with a margin of error of approximately four percentage points. Another method is bioelectrical impedance analysis (BIA), which uses the resistance of electrical flow through the body to estimate body fat. Another method is using a large tank of water to measure body buoyancy. Increased body fat will result in greater buoyancy, while greater muscle mass will result in a tendency to sink.

In another aspect, the present disclosure provides methods for treating an overweight or obese subject involving determining a level of at least one biomarker related to being overweight or obese in the subject, and administering an effective amount of a disclosed compound to achieve a target level in the subject. Exemplary biomarkers include body weight, Body Mass Index (BMI), Waist/Hip ratio WHR, plasma adipokines, and a combination of two or more thereof.

The pharmaceutically acceptable compositions of the present disclosure may be administered to patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a pharmaceutically acceptable composition of this present disclosure may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. A suitable treatment period can be, for example, at least about one week, at least about two weeks, at least about one month, at least about six months, at least about 1 year, or indefinitely. A treatment period can terminate when a desired result, for example a weight loss target, is achieved. A treatment regimen can include a corrective phase, during which dose sufficient to provide reduction of weight is administered, and can be followed by a maintenance phase, during which a e.g. a lower dose sufficient to prevent weight gain is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain body weight in subjects whose body weight has been previously controlled by other means, including diet and exercise, bariatric procedures such as bypass or banding surgeries, or treatments employing other pharmacological agents.

Another aspect of the present disclosure provides method of treating obesity in a patient in need thereof, comprising: administering a MetAP-2 inhibitor; monitoring the patient at discrete intervals for risk of a thromboembolic event; and determining at the discrete intervals that the patient has an indication identifying the patient is at particular risk of a thromboembolic event upon treatment with the MetAP-2 inhibitor.

In certain embodiments, monitoring may comprise administering a diagnostic test selected from the group consisting of: D-dimer test, prothrombin fragment 1.2, vWF level testing, presence/amount of vWF multimers, assessment of soluble P-selectin, assessment of pepin VWF, assessment of ADAMTS13 antigen, a fibrin degradation product test, a fibrin monomer test, clinical examination and scoring, a thrombophilia screening panel test, a thromoboelastogram (TEG), genetic testing, lower extremity ultrasound, a venogram, and/or a PE protocol CT scan.

For example, the clinical examination and scoring may comprise a Wells-score, patient history scoring, a MARNI (Marseilles-Nimes prediction model score for patient with known hereditary thrombophilia), TiC scoring, and/or Padua Prediction Score.

For example, the MetAP-2 inhibitor may be one of e.g., (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-morpholinoethyl)azetidine-1-carboxylate or a pharmaceutically acceptable salt or stereoisomer thereof.

A further aspect of the present disclosure provides a method of treating a non-oncologic disorder in a patient in need thereof, comprising administering to the patient a disclosed the pharmaceutically acceptable composition.

In certain embodiments, the non-oncologic disorder may be, for example, a metabolic disease. In certain other embodiments, the non-oncologic disorder may be, for example, obesity and/or a co-morbidity thereof. In further embodiments, the non-oncologic disorder may be, for example, chronic inflammatory disease or impaired wound healing.

In certain embodiments, the non-oncologic disorder may be, for example, a inflammatory disease. For example, the inflammatory disease may be selected from the group consisting of inflammatory bowel disease, Kawasaki disease, Sjogren's syndrome, systemic lupus erythematosus, rheumatoid arthritis, psoriatic arthritis, chronic obstructive pulmonary disease, and psoriasis.

Exemplary co-morbidities include cardiac disorders, endocrine disorders, respiratory disorders, hepatic disorders, skeletal disorders, psychiatric disorders, metabolic disorders, and reproductive disorders.

Exemplary cardiac disorders include hypertension, dyslipidemia, ischemic heart disease, cardiomyopathy, cardiac infarction, stroke, venous thromboembolic disease and pulmonary hypertension. Exemplary endocrine disorders include type 2 diabetes and latent autoimmune diabetes in adults. Exemplary respiratory disorders include obesity-hypoventilation syndrome, asthma, and obstructive sleep apnea. An exemplary hepatic disorder is nonalcoholic fatty liver disease. Exemplary skeletal disorders include back pain and osteoarthritis of weight-bearing joints. Exemplary metabolic disorders include Prader-Willi Syndrome and polycystic ovary syndrome. Exemplary reproductive disorders include sexual dysfunction, erectile dysfunction, infertility, obstetric complications, and fetal abnormalities. Exemplary psychiatric disorders include weight-associated depression and anxiety.

### Examples

The examples which follow are intended in no way to limit the scope of this disclosure but are provided to illustrate aspects of the present disclosure. Many other embodiments of this disclosure will be apparent to one skilled in the art.

### ADME and Pharmacokinetic Studies:

Compound A and beloranib are irreversible inhibitors of MetAP-2 of the fumagillin class. Although they both have the fumagillol-moiety that binds to MetAP-2 in their chemical structures, the different side chains are associated with significantly different physicochemical properties, as shown in Table 1. These differences likely contribute to both pharmaceutical and physiological differentiation for the two compounds. The absorption, distribution, metabolism, and excretion (ADME) characteristics of beloranib have been extensively studied. ADME data for Compound A demonstrated significant differences in how Compound A is absorbed, distributed, and cleared in non-clinical species and humans.

TABLE 1
Physicochemical Properties of Beloranib and Compound A

| Property | beloranib | Compound A |
|---|---|---|
| Molecular Weight | 499.64 | 478.30 |
| logD7.4 | 2.72 | 2.28 |
| pKa | 9.4 | 7.17 |
| Solubility (pH 7) | 0.3 mg/mL | >100 mg/mL |

The absorption, distribution, and metabolism (ADME) characteristics of Compound A are substantially different from those of beloranib for non-clinical species and humans. Following intravenous administration to rats and dogs, Compound A has a lower volume of distribution and higher clearance. Following subcutaneous administration to rats, dogs, and rabbits, Compound A has a much shorter Tₘₐₓ, higher Cₘₐₓ, and shorter half-life than beloranib, while AUC (area under the curve) values are similar for the two compounds. These same patterns observed in non-clinical species are observed for humans.

Reflecting these differences in pharmacokinetics, the plasma concentration profile for Compound A is compressed, relative to beloranib, so that the duration of exposure is much shorter. Notably in humans, Compound A concentrations in plasma after a 2.4 mg subcutaneous dose decreased to less than 2% of the Cₘₐₓ by four hours after dosing and were not quantifiable at 8 hours after dosing. In contrast, following a 2 mg subcutaneous dose, beloranib plasma concentrations decreased to approximately 50% of Cₘₐₓ by four hours after dosing and was still present (approximately 5.6% of Cₘₐₓ) at 24 hours after dosing.

In addition to these differences in pharmacokinetic profile, Compound A has distribution and metabolism characteristics which are distinctly different from beloranib. Plasma protein binding is much lower for Compound A (30-70% vs 95%), although red blood cell partitioning is similar for the two compounds.

Tissue distribution of Compound A and beloranib, in general, mirrors plasma concentrations. Thus, the duration of high levels of exposure to the tissues was much shorter for Compound A than for beloranib. Peak and residual radioactivity from beloranib in tissues was generally 2-5 fold higher than dose-normalized radioactivity in tissues from Compound A. Exceptions were that fat and bone were two tissues where radioactivity was higher for Compound A and kidney and liver were two tissues where radioactivity was similar for the two compounds.

Clear differences in the primary metabolic clearance pathways for Compound A and beloranib have been observed in hepatocyte incubation studies. Metabolic profiles of beloranib were similar in hepatocytes from all three species, with glutathione (GSH) conjugation pathways being predominant for all three species. Metabolic profiles of Compound A in hepatocytes were similar for dog and human with oxidative (CYP-mediated) metabolism being predominant. The metabolic profile of Compound A in rat hepatocytes demonstrated a higher amount of GSH-metabolism than for dog and human hepatocytes, but a much greater contribution of CYP-mediated metabolism was observed for Compound A in rat hepatocytes than for beloranib in rat hepatocytes.

Finally, reflecting the observations from the hepatocyte studies, the major metabolites observed in rats dosed SC with beloranib resulted from GSH conjugation and subsequent metabolic degradation of the GSH conjugates. The major metabolites observed in rats dosed SC with Compound A resulted from CYP-mediated conjugation.

Thus, the ADME characteristics of Compound A are different from those of beloranib. Differences in some of the fundamental processes (plasma protein binding and routes of metabolism) appear to lead to dramatic differences in tissue distribution (generally higher with beloranib) and pharmacokinetic profiles. These differences, which appear to be similar for non-clinical species and humans, lead to different profiles of exposure in plasma with subcutaneous dosing. Exposure of Compound A in humans is characterized by a rapid attainment of Cₘₐₓ and a subsequent rapid decrease of plasma concentrations, whereas exposure of beloranib in humans is characterized by a much slower absorption process with Cₘₐₓ attained much later, followed by slow decrease of plasma concentrations. It is anticipated that this “fast-in/fast-out” profile for Compound A will result in an improved safety and efficacy profile over beloranib.

Animal experiments were performed in accordance with the Guide for the Care and Use of Laboratory Animals as adopted and promulgated by the U.S. National Institutes of Health or local equivalent and were approved by the Institution's Animal Care and Use Committee or local equivalent. Unless described otherwise, animals (mouse, rat, and dog studies) were maintained under standard conditions (12-hour light/dark cycle and approximately 21±4° C. and 55±15% relative humidity), and food (Rodent pellet chow [Research Diets, Brunswick N.J., or similar] or canine pellet [Certified Dog Diet 2021C, Harlan Teklad Diets, Madison, Wis.] or canned [i.e., Cesar® Mars Petcare, Victoria, Australia]) and water were available ad libitum. Unless described otherwise, drug formulations were administered s.c. daily (efficacy studies) or every 3 days (Q3D; safety studies). Compound A vehicle was 5% mannitol in water (5% mannitol in 10 mM sodium phosphate, pH 7.5). Beloranib vehicle was 0.0416% sodium carboxymethyl cellulose, 0.0001% Poloxamer 188, and 0.1664% mannitol in 9.365 mM Sorenson buffer, pH 7.4.

Male and female beagle dogs of 9 to 11 months of age for PK characterization were individually housed at the testing facility Approximately 325 g of pelleted food was provided daily. Dogs received compound A 0.6 mg/kg s.c. (n=5 males and 5 females/group). In a separate study, dogs received beloranib 0.6 mg/kg s.c. (n=4 males and 4 females/group). Animals were observed twice daily for signs of ill health, morbidity, mortality, injury, and viability, and detailed clinical observations were conducted daily (including urine/fecal examination and hands-on examination). Food was weighed daily, and body weights were recorded at least weekly.

Whole venous blood samples of approximately 1.0 mL were collected from the jugular vein of animals on Day 1 at 0.25, 0.5, 1, 2, 4, and 8 hours after dosing (Compound A), or Day 1 at 0.5, 1, 2, 4, 8, 24, and 72 hours after dosing (beloranib). Samples were collected in tubes containing lithium heparin and processed for plasma by centrifugation (10 minutes at 3000 rpm). Supernatant was removed and stored frozen (−70° C.) until processed (BASi, West Lafayette, Ind.). Plasma drug concentration data were used to calculate PK parameters for individual concentration data using noncompartmental PK methods in Watson™ version 7.3.0.01 (Thermo Fisher Scientific, Inc., Waltham, Mass.). The lower limit of quantification (LLOQ) in dog plasma was 0.05 ng/mL for beloranib and 0.5 ng/mL for compound A.

### Example 1: Pharmacokinetics

The pharmacokinetic profiles following intravenous administration of beloranib or Compound A reflect differences in how each compound is distributed and cleared that are independent of pharmaceutical properties that affect absorption from subcutaneously administered compound. In rat and dog studies with intravenous administration, Compound A has a lower Vdₛₛ and a higher CL than beloranib, as shown in Table 2. Although Compound A has not been administered to humans intravenously, the rapid absorption profile observed with subcutaneous dosing permits estimation of Vdₛₛ and CL in humans for comparison with intravenous PK of beloranib, as shown in Table 3. Similar to non-clinical species, Compound A has a lower Vdₛₛ and a higher CL than beloranib. Thus, similar differences in the pharmacokinetic properties of Compound A and beloranib have been observed in non-clinical toxicology species and humans.

TABLE 2
Pharmacokinetics of Beloranib and Compound A in
Rats and Dogs After Intravenous Administration

| | | Dose | | | t_{1/2} | CL | Vdₛₛ |
|---|---|---|---|---|---|---|---|
| Compound | Species | (mg/kg) | Route | N | Hr | L/hr · kg | L/kg |
| beloranib | Rat | 3 | IV Bolus | 4 | 1.05 ± 0.23 | 6.77 ± 0.59 | 5.08 ± 0.29 |
| A | Rat* | 1 | IV Bolus | 4 | 1.55 ± 1.04 | 15.1 ± 0.5 | 2.11 ± 0.10 |
| beloranib | Dog | 0.142 | IV Bolus | 3 | 2.90 ± 0.29 | 2.00 ± 0.35 | 5.07 ± 0.23 |
| A | Dog | 1 | IV Bolus | 3 | 0.661 ± 0.101 | 2.72 ± 0.12 | 1.48 ± 0.17 |

*Pharmacokinetics (kₑₗ and dependent parameters) for the 1 mg/kg dose in rats with IV dosing were estimated using two time points (1 and 2 hours).

TABLE 3
Pharmacokinetics for Beloranib After Intravenous Administration and Compound A After Subcutaneous Administration

| | | Dose | | | kₑₗ | t_{1/2} | AUC∞ | CL | Vdₛₛ |
|---|---|---|---|---|---|---|---|---|---|
| Compound | Species | (mg) | Route | N | hr⁻¹ | hr | ng · hr/mL | L/hr · kg | L/kg |
| beloranib | Human | 2.0^{a} | IV Infusion | 9 | 0.120 ± 0.070 | 7.06 ± 2.66 | 5.82 ± 2.46 | 3.87 ± 1.82 | 25.4 ± 19.2 |
| A | Human | 2.4 | SC | 4 | 1.09 ± 0.34 | 0.682 ± 0.198 | 8.05 ± 1.42 | 5.07 ± 0.79 | 3.78 ± 1.54 |

^{a}The 30-minute IV infusion dose for beloranib was administered via surface area (0.9 mg/m²).

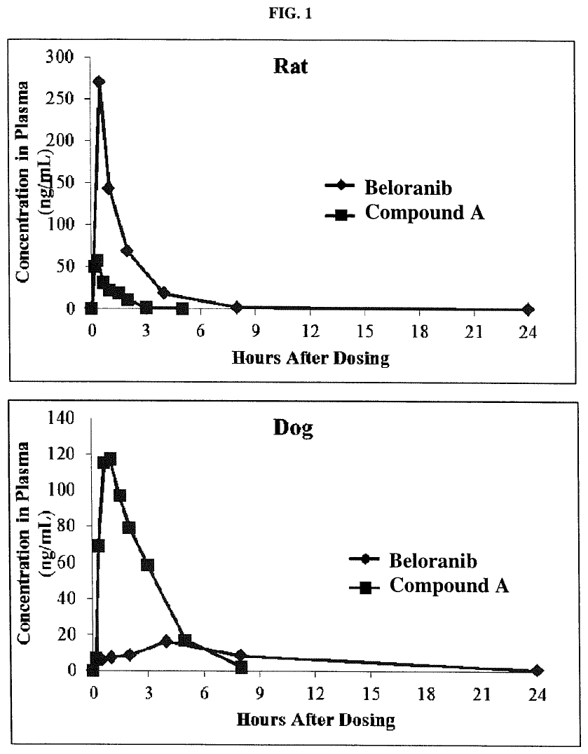

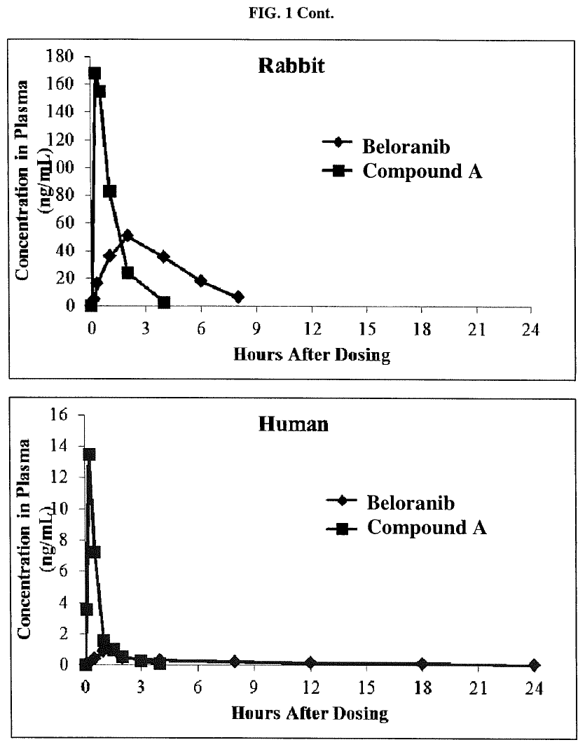

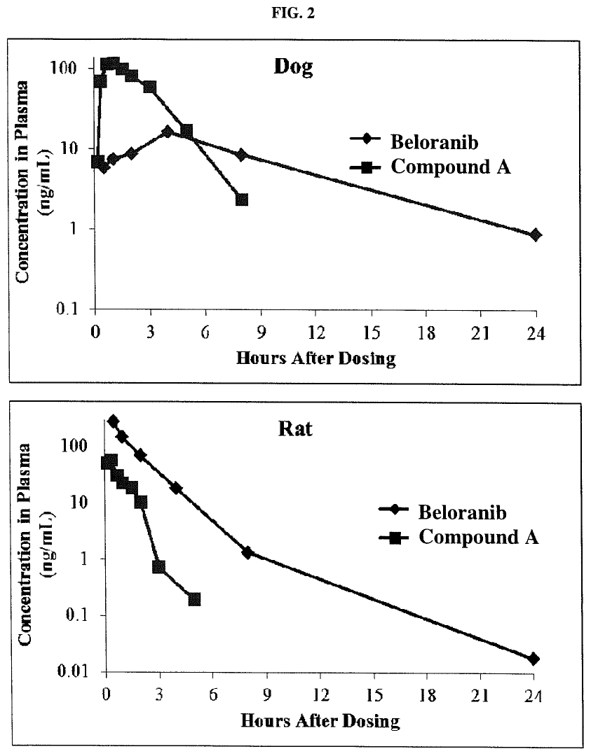

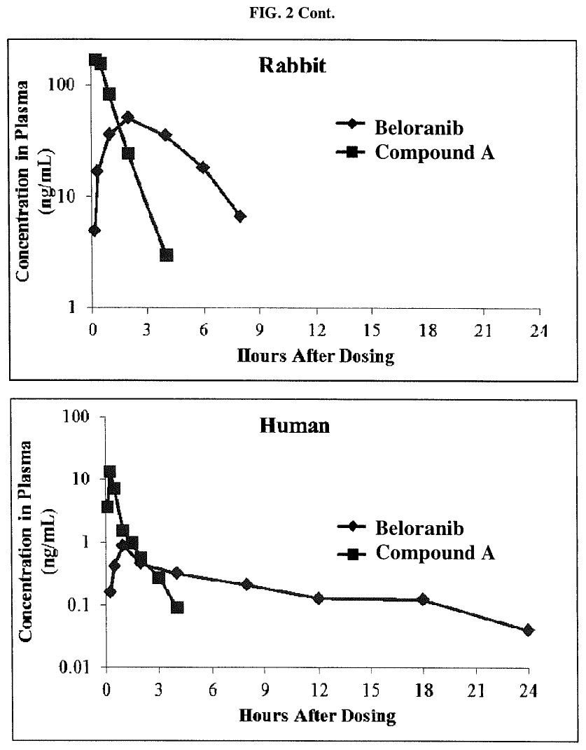

Assessment of differences in pharmacokinetics following subcutaneous administration reflect both differences in the pharmacokinetic properties of the compounds and differences in pharmaceutical factors that affect absorption of the formulations from the injection sites. In the three non-clinical species where pharmacokinetics following subcutaneous administration have been determined, a similar pattern of differentiation appears for each species, as shown in Table 4, FIG. 1, and FIG. 2. In all cases, Tₘₐₓ was marked shorter and Cₘₐₓ was much higher for Compound A, while AUC was similar (note: results for dog have not been normalized for dose). In addition, t_{1/2} was markedly shorter for Compound A. The same pattern was observed for humans, as shown in Table 5. The formulation of beloranib used for comparison with Compound A had a slightly faster SC release profile than the formulation used in later clinical studies, so comparisons between the two compounds were conservative and underestimated the differences.

TABLE 4
Pharmacokinetics of Beloranib and Compound A in Rat,
Rabbit, and Dog after Subcutaneous Administration

| | | Dose | | | Tₘₐₓ | Cₘₐₓ | AUCₗₐₛₜ | t_{1/2} |
|---|---|---|---|---|---|---|---|---|
| Compound | Species | (mg/kg) | Route | N | hr | ng/mL | ng · hr/mL | Hr |
| beloranib | Rat | 6 | SC | 3 | 0.5 | 270 | 412 | 1.8 |
| A | Rat* | 6 | SC | 4 | 0.291 ± 0.084 | 323 ± 26 | 391 ± 54 | 0.377 ± 0.0.042 |
| beloranib | Dog | 0.6 | SC | 4 | 3.50 ± 1.00 | 16.6 ± 12.3 | 182 ± 73 | 7.84 (N = 1) |
| A | Dog | 1 | SC | 4 | 0.917 ± 0.167 | 122 ± 29 | 348 ± 42 | 1.04 ± 0.30 |
| beloranib | Rabbit | 1 | SC | 3 | 1.7 ± 0.6 | 52.0 ± 4.1 | 222 ± 9 | 1.7 ± 0.3 |
| A | Rabbit | 1 | SC | 3 | 0.33 ± 0.14 | 173 ± 34 | 207 ± 45 | 0.615 ± 0.168 |

TABLE 5
Pharmacokinetics of Beloranib and Compound A in Humans after Subcutaneous Administration

| | | Dose | | | Tₘₐₓ | Cₘₐₓ | AUC∞ | kₑₗ | t_{1/2} |
|---|---|---|---|---|---|---|---|---|---|
| Compound | Species | (mg) | Route | N | hr | ng/mL | ng · hr/mL | hr⁻¹ | Hr |
| beloranib | Human | 2 | SC | 6 | 1.58 ± 1.28 | 1.0 ± 1.1 | 5.44 ± 1.81 | 0.0885 ± 0.0266 | 8.44 ± 2.49 |
| A | Human | 2.4 | SC | 4 | 0.25 ± 0.00 | 13.5 ± 2.7 | 8.05 ± 1.42 | 1.09 ± 0.34 | 0.682 ± 0.198 |

^{a}Interim data.

A notable difference between the pharmacokinetic profiles of beloranib and Compound A in all species was the prolonged exposure observed with beloranib, as shown in FIG. 2. While beloranib was quantifiable at 24 hours after dosing for rat, dog, and human, Compound A concentrations were below the lower limit of quantification within 6 to 8 hours after dosing. Even for rabbit (8 hours was the last plasma sampling time point for rabbit studies for both beloranib and Compound A) concentrations of plasma had dropped to below the lower limit of quantification by 4 hours, while beloranib was still quantifiable at 8 hours after dosing. With a dosing regimen of once every 3 days in animals and twice a week in humans, the interval between doses where concentrations of beloranib in plasma had decreased to background is approximately 48 hours (with a slightly longer average interval for humans with a slightly longer dosing interval). However, for Compound A, where concentrations in plasma return to baseline within 8 hours for all species, the interval between doses with minimal exposure is approximately 54 hours.

### Example 2: Distribution

Beloranib was extensively (>95%) bound to plasma proteins in blood from all non-clinical species tested and humans. However, Compound A was markedly less bound to plasma proteins (40-70% in rat and dog and 25-35% in human) and binding tended to decrease with increasing concentration, as shown in Table 6. The higher unbound fraction of Compound A may have contributed to the more rapid clearance of Compound A in pharmacokinetic studies, and to differences in tissue distribution. The tissue distribution of beloranib and Compound A was investigated in rats using radiolabeled compounds. Data from a long-term whole body autoradioluminography (WBAL) study with beloranib dosed subcutaneously at 1 mg/kg was compared with data from a short-term tissue dissection (TD) study with Compound A dosed subcutaneously at 6 mg/kg. For this assessment, the results from the Compound A study were normalized (assuming direct linearity) to a 1 mg/kg dose.

TABLE 6
Protein Binding of Beloranib and Compound A in
Plasma from Rat, Dog, Mouse, and Human Plasma
Beloranib

| | % Bound | | | | | | | |
|---|---|---|---|---|---|---|---|---|
| Concentration | Rat Plasma | | Dog Plasma | | Mouse Plasma | | Human Plasma | |
| (nM) | Mean | RSD | Mean | RSD | Mean | RSD | Mean | RSD |
| 30 | NA | NA | NA | NA | NA | NA | 94.92 | 0.13 |
| 60 | 97.15 | 0.84 | 94.80 | 1.51 | 98.19 | 0.28 | NA | NA |
| 120 | 94.50 | 0.82 | 94.78 | 1.32 | 98.09 | 0.2 | 94.56 | 0.78 |
| 240 | 95.85 | 0.44 | 95.34 | 0.27 | 97.41 | 0.53 | 95.20 | 0.48 |
| 480 | 96.74 | 0.35 | 95.50 | 0.9 | 97.78 | 0.3 | 95.28 | 0.24 |
| 1000 | 95.66 | 0.63 | 94.10 | 0.58 | 97.88 | 0.14 | 94.64 | 0.92 |
| All | 95.98 | 1.07 | 94.90 | 0.58 | 97.87 | 0.31 | 94.92 | 0.39 |

Compound A

| | % Bound | | | | | | | |
|---|---|---|---|---|---|---|---|---|
| Intended | Rat Plasma | | Dog Plasma | | Mouse Plasma | | Human Plasma | |
| Concentration | Actual | | Actual | | Actual | | Actual | |
| (nM)* | nM | Bound | nM | Bound | nM | Bound | nM | Bound |
| 5 | 3.64 | 67.7 | 3.17 | 68.9 | 5.20 | 70.2 | 1.49 | 33.6 |
| 10 | 7.60 | 71.0 | 6.05 | 59.4 | 11.1 | 70.7 | 3.14 | 31.9 |
| 50 | 39.7 | 57.4 | 32.0 | 67.4 | 56.4 | 69.8 | 15.5 | 31.2 |
| 100 | 76.0 | 53.4 | 59.1 | 59.7 | 112 | 66.7 | 32.7 | 30.6 |
| 500 | 370 | 54.9 | 320 | 54.3 | 496 | 62.3 | 165 | 29.6 |
| 1000 | 595 | 43.4 | 527 | 53.1 | 868 | 54.0 | 338 | 25.2 |

*Compound A concentrations decreased in both the plasma and buffer compartments during the 4-hour dialysis incubation time. The actual concentrations represented the concentrations at the end of the dialysis.

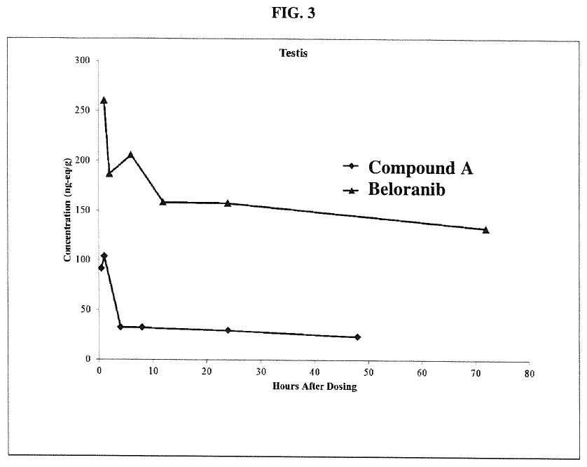

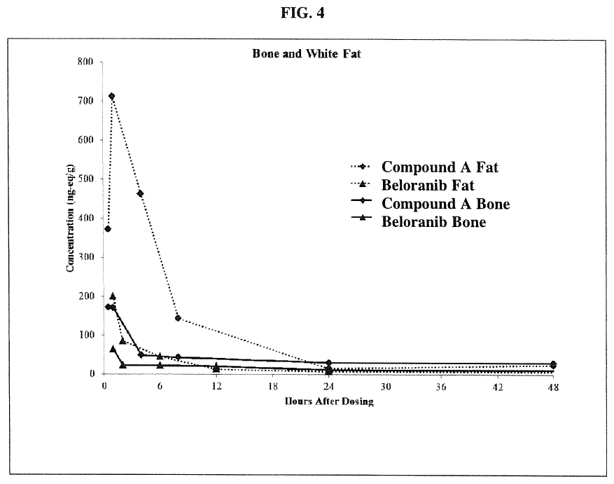

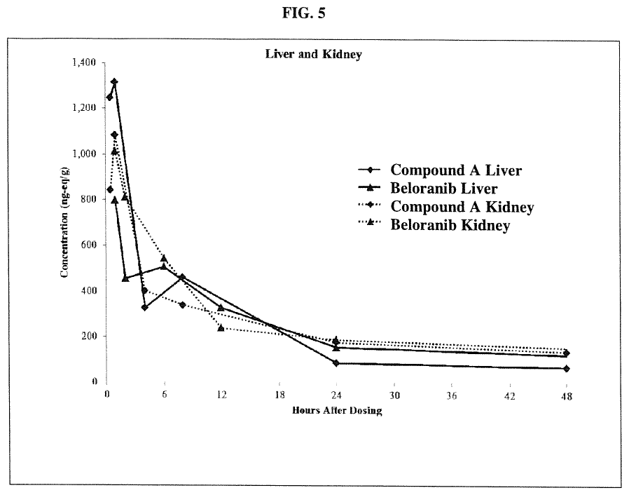

Tissue distribution of Compound A and beloranib, in general, mirrored plasma concentrations. Thus, the duration of high levels of exposure to the tissues were shorter for Compound A than for beloranib. Peak and residual radioactivity from beloranib in tissues was generally 2-5 fold higher than dose-normalized radioactivity in tissues from Compound A, as exemplified by testis and shown in FIG. 3. Exceptions were fat and bone tissues where radioactivity was higher for Compound A, as shown in FIG. 4, and liver and kidney tissues where radioactivity was similar for the two compounds, as shown in FIG. 5. In target engagement studies in a rat model, both compounds bound MetAP-2 effectively in target tissues, but the duration of exposure to free (not bound to MetAP-2) Compound A in those tissues was shorter than the duration of exposure to free beloranib.

### Example 3: Pharmacokinetic Studies in does

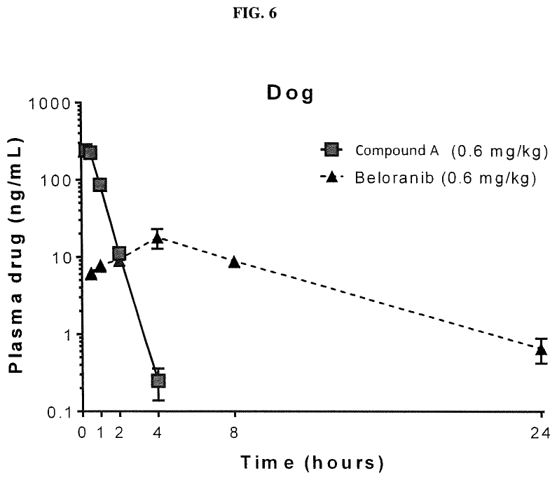

FIG. 6 shows plasma drug concentrations after a single s.c. injection in dogs (Day 1). In dogs, Compound A concentrations quickly reached the maximum observed concentration (Cmax) and rapidly declined to undetectable concentrations within a few hours after s.c. dosing (FIG. 6). The half-life (t½) of compound A was 0.4 hours in dogs. In contrast, the Cmax for beloranib occurred 4 or more hours after administration and beloranib plasma concentrations declined much more slowly. The t½ for beloranib was 9.3 hours in dogs.

### Example 4 Toxicology Studies in the Rat

Male and female Sprague Dawley rats 9-10 weeks of age (Envigo RMS, Inc., Indianapolis, Ind. or Orient Bio Inc., Gyeonggi-do, South Korea) were individually housed at the testing facility. Rats (n=15/group) received Compound A (2, 6, 25 mg/kg, or vehicle) Q3D for 28 days. In a separate study, rats (n=20/group) received beloranib (0.2, 0.6, 2, and 6 mg/kg, or vehicle [0.1% Tween 80/5% D-Mannitol solution]) Q3D for 28 days. Animals were allocated to treatment groups based on body weight, and body weight and food consumption were recorded weekly. In addition, animals were observed for clinical signs of toxicity, changes in body weight, as well as hematology, coagulation, and clinical chemistry parameters as described above. At necropsy, macroscopic pathologic findings were recorded, sperm motility and morphology were determined, selected organs were weighed, and selected tissues were collected for microscopic examination.

Table 7 illustrates safety comparisons between Compound A and beloranib administered s.c. Q3D for approximately 28 days in rat toxicology studies. For beloranib, doses ranging from 0.2-6 mg/kg were tested, where no observed adverse effect levels (NOAELs) of 0.6 (males) and 2 mg/kg (females) were established. At the highest dose, mortality was noted by Days 16-24, and this dose was decreased to 4 mg/kg. However, the dose was also not tolerated and the males in this group were terminated on Day 26. In contrast, Compound A doses ranged from 2-25 mg/kg, no mortality was noted, and the highest dose was well tolerated. The most notable finding was the increased degree of irritation and inflammation at the injection site in animals receiving the highest dose of compound a (25 mg/kg). After the 10-week recovery period, the lesions were mainly resolved with generally minimal fibrosis noted in 3/10 rats. Other findings of generally minimal grade and low incidence were mixed cell inflammation in lung (minimal to mild), and minimal individual hepatocellular necrosis (that resolved after dosing was stopped). With respect to exposure at the NOAEL, Cₘₐₓ and area under the curve from time 0 to the last measurable timepoint (AUC₀₋ₜ) were markedly higher with Compound A compared to beloranib. For example, the repeat dose AUC₀₋ₜ for males and females was 409 and 288 ng×h/mL for Compound A, respectively, vs 29.0 and 56.7 ng×h/mL for beloranib, respectively, indicating exposures ranging from 5- to 14-fold higher at the NOAEL for Compound A. Greater exposure differences for Compound A vs beloranib were observed for the Cₘₐₓ (21- to 52-fold).

TABLE 7
4 weeks of exposure

| | A | Beloranib |
|---|---|---|
| Dose (s.c., Q3D) | 0, 2, 6, and 25 mg/kg | 0, 0.2, 0.6, 2, and 6^{a} mg/kg |
| NOAEL | 6 mg/kg (M, F) | 0.6 mg/kg (M), 2 mg/kg (F) |
| Mortality/early euthanasia | None | 4/15 males in the 6 mg/kg dose |
| | | group died by Day 24, and the |
| | | remaining males in the 6 mg/kg |
| | | group were euthanized on Day 26 |

Histopathology

| | | |
|---|---|---|
| Target organs: finding, grade (dose, | Treatment site: irritation, | Spleen: decreased cellularity, |
| mg/kg) | inflammation, seroma formation | minimal (≥2 mg/kg) |
| | (25 mg/kg) | Bone marrow: decreased |
| | Lung: mixed cell inflammation, | cellularity, minimal to moderate |
| | mild (25 mg/kg) | with increased fat (≥2 mg/kg) |
| | Liver: necrosis, minimal | Thymic atrophy: lymphoid |
| | (25 mg/kg) | depletion, minimal to mild |
| | | (≥2 mg/kg) |
| | | Testes: germ cell |
| | | depletion/degeneration and marked |
| | | Leydig cell atrophy, minimal to |
| | | severe (≥2 mg/kg) |

Exposure

| | | |
|---|---|---|
| AUC₀₋ₜ at NOAEL (ng × hr/mL) | | |
| Day 1 | 349 (M), 262 (F) | 36.8 (M), 96.9 (F) |
| Day 28/31^{b} | 409 (M), 288 (F) | 29.0 (M), 56.7 (F) |
| Cₘₐₓ at NOAEL (ng/mL) | | |
| Day 1 | 351 (M), 278 (F) | 19.2 (M), 28.5 (F) |
| Day 28/31^{b} | 426 (M), 412 (F) | 8.17 (M), 19.2 (F) |

Male and female rats were dosed with Compound A(n = 15/group) or beloranib (n = 20/group).
^{a}Dose decreased from 6 to 4 mg/kg after mortality was observed in male rats during Days 16 to 24.
^{b}Samples collected on Day 28 (Compound A) or Day 31 (beloranib).

### Example 5 Metabolism

Clear differences in the primary metabolic clearance pathways for Compound A and beloranib were observed in hepatocyte incubation studies. Metabolic profiles of beloranib were similar in hepatocytes from all three species, with glutathione (GSH) conjugation pathways being predominant for all three species. Metabolic profiles of Compound A in hepatocytes were similar for dog and human with oxidative (CYP-mediated) metabolism being predominant. The metabolic profile of Compound A in rat hepatocytes demonstrated a higher amount of GSH-metabolism than for dog and human hepatocytes, but a much greater contribution of CYP-mediated metabolism was observed for Compound A in rat hepatocytes than for beloranib in rat hepatocytes.

Consistent with the observations from the hepatocyte studies, the major metabolites observed in rats dosed SC with beloranib resulted from GSH conjugation and subsequent metabolic degradation of the GSH conjugates. The major metabolites observed in rats dosed SC with Compound A resulted from CYP-mediated oxidation.

A clinical study identifying human circulating metabolites of Compound A was undertaken. Plasma samples from a cohort of six human subjects receiving administration of Compound A (4.8 mg) were pooled from all six subjects. The plasma samples were analyzed by LC/MS-MS for the parent compound, fumagillol, and other “key” metabolites (e.g., those with peak areas approaching 5-10% of total peak area). The most abundant metabolites were then semi-quantitated in pooled plasma samples.

A representative metabolite identified in the above study is shown below. It will be appreciated by a person of skill in the art that for each structure shown additional diastereomers and/or enantiomers may be envisioned, each of which is contemplated herein.

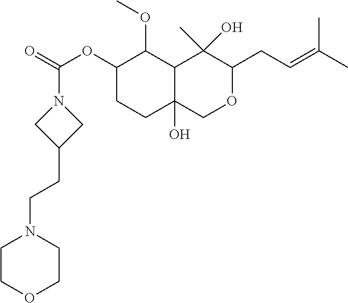 [SMILES: COC1C(OC(=O)N2CC(CCN3CCOCC3)C2)CCC2(O)COC(CC=C(C)C)C(C)(O)C12]

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A pharmaceutically acceptable composition comprising a MetAP-2 inhibitor, and a pharmaceutically acceptable excipient, said composition providing at least one of: a volume of distribution at steady state (VDₛₛ) of less than about 5 L/kg, a Tₘₐₓ of less than about 0.5 hr and/or Cₘₐₓ of more than about 10 ng/mL upon subcutaneous administration to a human patient.

2. The composition of claim 1, wherein the composition provides a half life (t_{1/2}) of less than 1 hour upon subcutaneous administration to the patient.

3. The composition of claim 1, wherein the composition provides less than a plasma concentration of 1 ng/mL about 8 hours after the administration.

4. The composition of claim 1 or 2, wherein the composition provides less than a plasma concentration of less than about 0.15 ng/mL about 8 hours after the administration.

5. The composition of any one of claims 1-4, wherein the composition provides a plasma concentration of less than the EC₅₀ of the METAP-2 inhibitor at about 8 hours after administration.

6. The composition of claim 5, wherein the EC₅₀ of the METAP-2 inhibitor is measured in a HUVEC cell at 72 hours.

7. A pharmaceutically acceptable composition comprising a MetAP-2 inhibitor and a pharmaceutically acceptable excipient, wherein the half-life of the MetAP-2 inhibitor upon subcutaneous administration to a human patient is about 10 times less than the half-life of beloranib when intravenously administered to a human patient.

8. The pharmaceutically acceptable composition of any one of claims 1-7, wherein the MetAP-2 inhibitor is an irreversible inhibitor.

9. The pharmaceutically acceptable composition of claim 8, wherein the irreversible inhibitor covalently bonds to His231 of MetAP-2 via a spiro epoxide moiety present on the irreversible inhibitor.

10. The pharmaceutically acceptable composition of claim 9, wherein the MetAP-2 inhibitor is an analog of fumigillin.

11. The pharmaceutically acceptable composition of claim 10, wherein the MetAP-2 inhibitor is represented by: 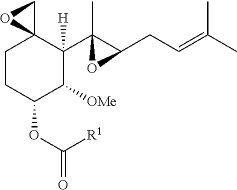
wherein R¹ is selected from C₁₋₈alkylene, C₂₋₈alkenylene, heterocyclic, C₃₋₆cycloalkyl, —NR^{a}—C₁₋₈alkylene, —NR^{a}—C₂₋₈alkenylene, and —NR^{a}—C₃₋₆cycloalkyl wherein R¹ is substituted by a substituent selected from the group consisting of: carboxy, —C(O)—O—C₁₋₆alkyl, —O—C(O)—NR^{a}R^{b}, phenyl (optionally substituted by substituent selected from NR^{a}R^{b}), C₁₋₆alkoxy (optionally substituted by a substituent selected from the group consisting of NR^{a}R^{b}, C₁₋₆alkyl, and heterocyclic)), C₁₋₆alkylene (optionally substituted by halogen, hydroxyl, heterocyclic, NR^{a}R^{b}, carboxy, —O—C(O)—NR^{a}R^{b}, and —C(O)—O—C₁₋₆alkyl), wherein R^{a} and R^{b} are each independently selected from hydrogen, and C₁₋₆alkyl, or together with the nitrogen to which they are attached form a 4-7 membered heterocyclic ring;
and pharmaceutically acceptable salts, stereoisomers, esters and prodrugs thereof.

12. The pharmaceutically acceptable composition of any one of claims 1-11, wherein the MetAP2 inhibitor is represented by Formula I: 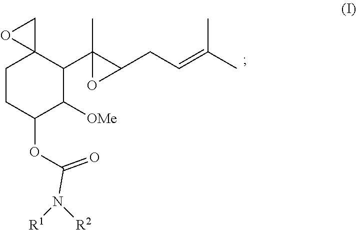
wherein:
R¹ and R², together with the nitrogen to which they are attached, form a 4-6 membered saturated heterocyclic ring A or form a 6-8 membered bicyclic, fused, bridged or spirocyclic hetercyclic ring A, which may have an additional heteroatom selected from the group consisting of O, S(O)_{w} (wherein w is 0, 1, or 2), and NR^{a};
heterocyclic ring A is substituted on an available carbon by a substituent represented by L-B; and wherein heterocyclic ring A is additionally and optionally substituted by one or two substituents each independently selected from the group consisting of halogen, hydroxyl, C₁₋₃alkyl and C₁₋₃alkoxy; wherein C₁₋₃alkyl and C₁₋₃alkoxy may optionally be substituted by one or more fluorine atoms or a substituent selected from the group consisting of cyano, hydroxyl, and N(R^{a}R^{b});
L is selected from the group consisting of C₁₋₆alkylene and C₁₋₆alkenylene; wherein C₁₋₆alkylene and C₁₋₆alkenylene may optionally be substituted by one or two substituents each independently selected from the group consisting of halogen and hydroxyl; and wherein one or two methylene units of L may optionally and independently be replaced by a moiety selected from the group consisting of a bond, —O—, —C(O)—, —O—C(O)—, —C(O)—O—, —NR^{a}—, —C(O)—NR^{a}—, —NR^{a}—C(O)—, —O—C(O)—NR^{a}—, —NR^{a}—C(O)—O—, —S(O)_{w}— (wherein w is 0, 1, or 2), —S(O)_{w}—NR^{a}—, and —NR^{a}—S(O)_{w}—;
B is selected from the group consisting of RⁱR^{j}N—, heterocyclyl, heterocyclyloxy, heteroaryl, heterocyclyl-(NR^{a})—, and hydrogen; wherein said heteroaryl may optionally be substituted with one or more substituents selected from R^{f}; and wherein said heterocyclyl is bound to L through a ring carbon and may optionally be substituted by one or more substituents selected from R^{g}; and wherein if said heterocyclyl contains a —NH moiety that nitrogen may optionally be substituted by R^{h};
Rⁱ and R^{j} are selected independently for each occurrence from the group consisting of hydrogen, C₁₋₆alkyl, C₂₋₆alkenyl, C₃₋₆cycloalkyl, heterocyclyl and heterocyclylcarbonyl; wherein C₁₋₆alkyl, C₂₋₆alkenyl and C₃₋₆cycloalkyl may be optionally substituted by one or more substituents independently selected from the group consisting of fluorine, hydroxyl, cyano, R^{a}R^{b}N—, R^{a}R^{b}N-carbonyl- and C₁₋₃alkoxy; and wherein heterocyclyl and heterocyclylcarbonyl may be optionally substituted by one or more substituents independently selected from the group consisting of C₁₋₆alkyl, C₂₋₆alkenyl, C₂₋₆alkynyl, C₃₋₆cycloalkyl, C₁₋₆alkoxy, halo-C₁₋₆-alkyl, hydroxyl-C₁₋₆-alkyl, R^{a}R^{b}N—C₁₋₆alkyl- and C₁₋₆-alkoxy-C₁₋₆-alkyl group; and wherein if said heterocyclyl or heterocyclylcarbonyl contains a —NH moiety that nitrogen may optionally be substituted by one or more groups independently selected from the group consisting of C₁₋₆alkyl, C₃₋₆alkenyl, C₃₋₆alkynyl, C₃₋₆cycloalkyl, C₁₋₆alkyl-S(O)₂— and C₁₋₆-alkylcarbonyl;
or Rⁱ and R^{j} taken together with the nitrogen to which they are attached form a 4-9 membered monocyclic, bridged bicyclic, fused bicyclic or spirocyclic heterocyclic ring, which may have an additional heteroatom selected from the group consisting of O, N, and S(O)_{w} (wherein w is 0, 1 or 2); wherein the 4-9 membered monocyclic, bridged bicyclic, fused bicyclic or spirocyclic heterocyclic ring may be optionally substituted on carbon by one, two, or more substituents selected from the group consisting of halogen, hydroxyl, oxo, cyano, C₁₋₆alkyl, C₁₋₆alkoxy, R^{a}R^{b}N—, R^{a}R^{b}N—SO₂— and R^{a}R^{b}N-carbonyl-; wherein said C₁ 6alkyl or C₁₋₆alkoxy may optionally be substituted the group consisting of fluorine, hydroxyl, and cyano; and wherein if said 4-9 membered monocyclic, bridged bicyclic, fused bicyclic or spirocyclic heterocyclic ring contains a —NH moiety that nitrogen may be optionally substituted by a substituent selected from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, and C₁₋₆alkoxycarbonyl- may optionally be substituted by one or more substituents selected from the group consisting of fluorine, hydroxyl, and cyano;
R^{a} and R^{b} are independently selected, for each occurrence, from the group consisting of hydrogen and C₁₋₃alkyl; wherein C₁₋₃alkyl may optionally be substituted by one or more substituents selected from halogen, cyano, oxo and hydroxyl;
R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, (wherein wherein w is 0, 1 or 2), C₁₋₆alkylcarbonyl-N(R^{a})— and C₁₋₆alkoxycarbonyl-N(R^{a})—; wherein C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, C₁₋₆alkoxycarbonyl-N(R^{a})— may be optionally substituted by one or more substituents selected from R^{P};
R^{g} is independently selected for each occurrence from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}— (wherein w is 0, 1 or 2), C₁₋₆alkylcarbonyl-N(R^{a})— and C₁₋₆alkoxycarbonyl-N(R^{a})—; wherein C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})— may be optionally substituted by one or more substituents selected from R^{P};
R^{h} is independently selected for each occurrence from the group consisting of hydrogen, C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, C₁₋₆alkoxycarbonyl-, RⁱR^{j}N-carbonyl- and RⁱR^{j}N—SO₂—; wherein C₁₋₆alkyl, C₃₋₆alkenyl, C₃₋₆alkynyl, C₃₋₆cycloalkyl, C₁₋₆alkyl-S(O)₂—, C₁₋₆alkylcarbonyl-, and C₁₋₆alkoxycarbonyl- may optionally be substituted by one or more substituents selected from R^{P}; and
R^{P} is independently selected, for each occurrence, from the group consisting of halogen, hydroxyl, cyano, C₁₋₆alkoxy, RⁱR^{j}N—, RⁱR^{j}N-carbonyl-, RⁱR^{j}N—SO₂—, and RⁱR^{j}N-carbonyl-N(R^{a})—;
and pharmaceutically acceptable salts, stereoisomers, esters and prodrugs thereof.

13. The pharmaceutically acceptable composition of any one of claims 1-12, wherein the MetAP-2 inhibitor is (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-morpholinoethyl)azetidine-1-carboxylate or a pharmaceutically acceptable salt or stereoisomer thereof.

14. The pharmaceutically acceptable composition of any one of claims 1-13, wherein upon administration, the MetAP-2 inhibitor degrades to metabolites resulting from one or more metabolic pathways selected from the group consisting of CYP-mediated oxidation, GSH conjugation, and epoxide hydrolase-mediated hydrolysis.

15. A compound represented by: 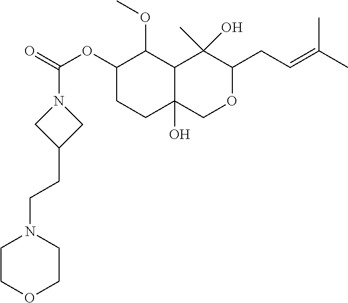 [SMILES: COC1C(OC(=O)N2CC(CCN3CCOCC3)C2)CCC2(O)COC(CC=C(C)C)C(C)(O)C12]
or a pharmaceutically acceptable salt or stereoisomer thereof.

16. A method of treating obesity in a patient in need thereof, comprising administering the pharmaceutically acceptable composition of any one of claims 1-15.

17. The method of claim 16, wherein the composition is administered daily, every other day, or two or three times a week.

18. A method of treating obesity in a patient in need thereof, comprising:
administering a MetAP-2 inhibitor;
monitoring the patient at discrete intervals for risk of a thromboembolic event; and
determining at the discrete intervals that the patient has an indication identifying the patient is at particular risk of a thromboembolic event upon treatment with the MetAP-2 inhibitor.

19. The method of claim 18, wherein monitoring comprises administering a diagnostic test selected from the group consisting of: D-dimer test, prothrombin fragment 1.2, vWF level testing, presence/amount of vWF multimers, assessment of soluble P-selectin, assessment of pepin VWF; assessment of ADAMTS13 antigen, a fibrin degradation product test, a fibrin monomer test, clinical examination and scoring, a thrombophilia screening panel test, a thromoboelastogram (TEG); genetic testing, lower extremity ultrasound, a venogram, and/or a PE protocol CT scan.

20. The method of claim 19, wherein the clinical examination and scoring comprises a Wells-score, patient history scoring, a MARNI (Marseilles-Nimes prediction model score for patient with known hereditary thrombophilia, TiC scoring, and/or Padua Prediction Score.

21. The method of any one of claims 18-20, wherein the MetAP-2 inhibitor is (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-morpholinoethyl)azetidine-1-carboxylate or a pharmaceutically acceptable salt or stereoisomer thereof.

22. A method of treating a non-oncologic disorder in a patient in need thereof, comprising administering the pharmaceutically acceptable composition of any one of claims 1-15.

23. The method of claim 22, wherein the non-oncologic disorder is a metabolic disease.

24. The method of claim 22 or 23, wherein the non-oncologic disorder is obesity and/or a co-morbidity thereof.

25. The method of any one of claims 22-24, wherein the non-oncologic disorder is type 2 diabetes or latent autoimmune diabetes.

26. The method of claim 22, wherein the non-oncologic disorder is chronic inflammatory disease or impaired wound healing.

27. The method of claim 22, wherein the non-oncologic disorder is an inflammatory disease.

28. The method of claim 27, wherein the inflammatory disease is selected from the group consisting of inflammatory bowel disease, Kawasaki disease, Sjogren's syndrome, systemic lupus erythematosus, rheumatoid arthritis, psoriatic arthritis, chronic obstructive pulmonary disease, and psoriasis.
